(19) United States
(12) Patent Application Publication
(10) Pub. No.: US 2021/0290609 A1
(43) Pub. Date: Sep. 23, 2021

(54) DEPOT ADMINISTRATION OF ILOPERIDONE

(71) Applicant: Vanda Pharmaceuticals Inc., Washington, DC (US)

(72) Inventor: Mihael H. Polymeropoulos, Potomac, MD (US)

(21) Appl. No.: 17/053,870

(22) PCT Filed: Dec. 4, 2019
(86) PCT No.: PCT/US2019/064401

§ 371 Date: Nov. 9, 2020

Related U.S. Application Data
(60) Provisional application No. 62/774,979, filed on Dec. 4, 2018.

(51) Int. Cl. A61K 31/454; A61K 9/00; A61K 9/10

(52) U.S. Cl. CPC A61K 31/454; A61K 9/10; A61K 9/0024

(57) ABSTRACT

Methods of preparing and administering an injectable depot formulation of crystalline iloperidone are disclosed herein.

### CROSS REFERENCE TO RELATED APPLICATIONS

The present application claims the benefit of U.S. Provisional Application No. 62/774,979, filed Dec. 4, 2018.

### BACKGROUND OF THE INVENTION

The present invention relates generally to methods of preparation and administration of a depot formulation of the atypical antipsychotic, iloperidone, and more particularly, to methods for preparing and administering a suspension of crystalline iloperidone.

Iloperidone (1-[4-[3-[4-(6-flouro-1,2-benzisoxazol-3-yl)-1-piperidinyl]propoxy]-3-methoxyphenyl]ethanone) is an atypical antipsychotic disclosed in U.S. Pat. RE39198, and described therein as being useful as an antipsychotic and an analgesic. Iloperidone is approved for use in the US in the treatment of schizophrenia. Schizophrenia is a severe form of long-term, chronic mental illness. Treatment of schizophrenia typically includes the continuous, long-term use of antipsychotic medication to effectively maintain control of symptoms and prevent relapse. Patient adherence to a prescribed long-term drug treatment regimen is acknowledged to be one of the most significant challenges in the treatment of schizophrenia.

To improve patient compliance, efforts have been made to develop controlled release depot formulations of antipsychotic drugs such as iloperidone. For example, microencapsulated depot formulations of iloperidone and a polylactide-co-glycolide polymer are described in U.S. Pat. Nos. 7,767,230 and 8,815,293. Further, an injectable depot formulation comprising crystals of iloperidone or its metabolite suspended in an aqueous vehicle, in which the release and absorption of the crystals in plasma can be correlated with the size of the crystals, is described in U.S. Pat. Nos. 8,293,765; 8,227,488; and 8,614,232.

One of the challenges associated with intramuscular injection of a depot formulation is clogging of the needle during the injection. Such clogging is particularly associated with depot formulations that are pre-mixed or pre-constituted.

### BRIEF DESCRIPTION OF THE INVENTION

Various aspects of the invention disclosed herein relate to methods of preparing and administering an injectable depot formulation of crystalline iloperidone suspended in a vehicle.

In a first aspect, a method is provided for preparing an injectable depot formulation of crystalline iloperidone, by combining crystalline iloperidone with a suspension vehicle to produce a suspension having a concentration of 166.67 mg to 200 mg of crystalline iloperidone per mL of vehicle solution. The crystalline iloperidone may be suspended in the vehicle, e.g., by agitating, vortexing, or manually shaking. The suspension vehicle in which the crystals are suspended may be, e.g., an aqueous solution, and the crystalline iloperidone may be characterized by a Dv50 of up to about 120 μm, about 91 μm to about 118 μm, or about 98 μm to about 105 μm.

In a second aspect, a method is provided for administering an injectable depot formulation of crystalline iloperidone suspended in a vehicle. The method includes, using a syringe, intramuscularly (IM) injecting the depot formulation in a single, fast push of the syringe plunger. For example, a suspension volume of about 2.5 mL to about 3.0 mL, or about 1.25 mL to about 1.5 mL, is administered via IM injection over a period of about five (5) seconds or less. The depot formulation may further contain crystalline iloperidone characterized by a Dv50 of up to about 120 μm, about 91 μm to about 118 μm, or about 98 μm to about 105 μm, and vehicle in a concentration of about 166.67 mg to about 200 mg crystalline iloperidone per mL of vehicle. The administration may be performed less than 24 or less than 48 hours after the crystals are suspended in the vehicle.

In a third aspect, a method is provided for preparing and administering an injectable depot formulation of crystalline iloperidone. According to this method, crystalline iloperidone, which may be characterized by a Dv50 of up to about 120 μm, about 91 μm to about 118 μm, or about 98 μm to about 105 μm, is combined with a suspension vehicle in a concentration of 166.67 mg to 200 mg of crystalline iloperidone per mL of vehicle solution. The crystalline iloperidone may be suspended in the vehicle, e.g., by agitating, vortexing, or manually shaking. The suspension vehicle in which the crystals are suspended may be, e.g., an aqueous solution. Following suspension, the depot formulation is then administered by intramuscularly (IM) injecting the depot formulation with a syringe, in a single, fast push of the syringe plunger. For example, a suspension volume of about 2.5 mL to about 3.0 mL is administered via IM injection over a period of about five (5) seconds or less. The administration may be performed less than 24 or less than 48 hours after the crystals are suspended in the vehicle.

In a fourth aspect, an injectable depot formulation of crystalline iloperidone is provided herein, that is prepared by the processes described herein, for example, those described above in the first aspect or elsewhere hereinbelow.

These and other aspects, advantages and salient features of the invention will become apparent from the following detailed description, which discloses embodiments of the invention.

### DETAILED DESCRIPTION OF THE INVENTION

In various embodiments of the invention, methods described herein include methods of preparing an injectable depot formulation of crystalline iloperidone, products prepared according to these processes, and methods for administering an injectable depot formulation of crystalline iloperidone. The crystalline iloperidone used in the methods described herein is known in the art or can be prepared by known methods, see e.g., U.S. Pat. Nos. 8,293,765, 8,227,488, and 8,614,232, describing iloperidone crystals with a D₅₀ which may be from about 1 to about 200 μm, from about 10 to about 170 μm, or from about 15 to about 70 μm.

The iloperidone crystals for suspension may be in the form of needles, trigonal forms, tetragonal forms, flat rod shapes, cubes, parallelepipeds, or may be plate-like. The particle size distribution of the crystals may be characterized by a number of measures, such as Dv10, Dv50, and Dv90. In this context, Dv10, Dv50, and Dv90 have their customary meanings as understood by the skilled individual, i.e., the Dv50 value represents the median particle size by volume, or the maximum particle diameter below which 50% of the sample volume exists. The Dv10 value represents the maximum particle diameter below which 10% of the sample volume exists, and the Dv90 value represents the maximum particle diameter below which 90% of the sample volume exists. In embodiments of the present invention, the iloperidone crystals may be characterized by a Dv50 which may be up to about 120 μm, about 91 μm to about 118 μm, or about 98 μm to about 105 μm. In addition to Dv50, the crystals of iloperidone may be further characterized by Dv10 and Dv90, for example, a Dv10 of about 14 μm to about 50 μm, or about 22 μm to about 29 μm; and a Dv90 of about 188 μm to about 241 μm, or about 174 μm to about 180 μm. The foregoing particle sizes may be determined using a method as described herein in Example 1. The modifier “about,” as used in connection with a quantity is inclusive of the stated value and has the meaning dictated by the context, for example, “about 180 μm” includes 180 μm±the degree of error associated with measurement of the particular quantity. The crystalline iloperidone may be contained in a single unit dosage form, e.g., a vial, and may contain about 600 mg of crystalline iloperidone.

In one embodiment, a method is provided herein for preparing an injectable depot formulation of the above-described crystalline iloperidone, resulting in proper reconstitution of the crystalline iloperidone for intramuscular injection. According to the preparation methods provided herein, the crystalline iloperidone is combined with a suspension vehicle.

In various embodiments, the suspension vehicle includes a wetting agent, a viscosity enhancer, an osmotic agent, a solvent, and may include a processing gas. In particular, the wetting agent may be Poloxamer 188, which may be present in an amount of 4.00 mg/2 mL; the viscosity enhancer may be carboxymethyl cellulose (CMC) sodium, which may be present in an amount of 14.00 mg/2 mL; the osmotic agent may be mannitol, which may be present in an amount of 90.00 mg/2 mL; the solvent may be water, which may be present in an amount of q.s. to 2 mL (not including 0.2 mL overfill); and the processing gas may be nitrogen, which may be present q.s. The processing gas may be omitted or may be removed during processing prior to suspension of the crystalline iloperidone.

The combination of the crystalline iloperidone and vehicle may be accomplished, for example, by withdrawing, via a syringe, a suitable volume of vehicle from one or more vehicle ampoules. For example, about 3.0 mL to about 3.6 mL, e.g. 3.3 mL or 3.4 mL of vehicle is used to suspend 600 mg of crystalline iloperidone. If necessary, excess vehicle volume and air are removed from the syringe to waste, and the syringe barrel may be tapped if necessary.

As noted, the crystalline iloperidone may be contained in a vial, which may be positioned at an angle of approximately 45° with respect to the counter or table surface. The edge of the vial is firmly tapped against the surface, e.g. approximately four times, to allow the crystals to flow. The vial is then rotated approximately one third of a turn and the tapping process is repeated. The turning and tapping process may be completed about three to about five times over a period of 15 to 30 seconds, or until the majority of crystals in the vial are free flowing.

The syringe containing the desired volume of vehicle is used to slowly inject the vehicle into the vial containing the crystalline iloperidone, wetting all the walls of the vial in the process. After combining the crystalline iloperidone with the suspension vehicle, e.g., by injecting as described above, the crystalline iloperidone may be suspended in the suspension vehicle by agitating, vortexing, manually mixing, or shaking the vial containing the crystalline iloperidone and vehicle. The agitating, vortexing, manual mixing, or shaking may be performed for about 30 seconds or longer, e.g., about 60-90 seconds. If a visual check indicates that the crystalline iloperidone is not completely suspended, e.g., powder residue remains at the base of the vial or the suspension does not appear uniform, the agitating, vortexing, manual mixing, or shaking is repeated.

If the visual check indicates that the crystalline iloperidone is completely suspended, the suspension may optionally sit undisturbed for a fifteen (15) minute period. After the expiration of the 15 minute period, the suspension may be gently re-dispersed, e.g. by slowly turning the vial upside down for 10-15 seconds, without agitating, vortexing, manual mixing, or shaking.

Following suspension of the crystals in vehicle as described above, the crystalline iloperidone and the suspension vehicle may be present in the resulting suspension at a concentration of about 166.67 mg to about 200 mg of crystalline iloperidone per mL of vehicle solution. This concentration may be the result of suspending 600 mg of crystalline iloperidone in 3.0 to 3.6 mL, or may be the result of suspending a larger or smaller dose of crystalline iloperidone in a larger or smaller (respectively) vehicle volume. 600 mg of crystalline iloperidone in 3.0 to 3.6 mL of vehicle provides suitable fill for a 500 mg dose of iloperidone after accounting for overage that remains in vessels, e.g. in a vial, syringe barrel, or needle, during preparation and administration. For smaller desired doses of iloperidone such as, e.g., 125 to 500 mg, 125-250 mg, or about 250 mg, the amount of iloperidone and volume of vehicle used may be reduced while maintaining the proportions described herein.

Following the suspension of the crystals in the vehicle, regardless of whether the optional 15 minute rest period and subsequent gentle re-dispersion steps are carried out, a dosage of the suspension is promptly drawn into a syringe for administration to a subject. In methods including the optional 15 minute rest period and gentle re-dispersion step, the dosage of suspension may be drawn into the syringe, e.g. within about 20 seconds of completion of the gentle re-dispersion step.

The desired volume of suspension contained in the syringe depends upon the exact dosage being administered. In one example, a 500 mg crystalline iloperidone dose is desired, contained in a suspension volume between about 2.5 mL and about 3.0 mL. This may be achieved by drawing 2.5 to 3.0 mL, e.g., about 2.8 mL of suspension into the syringe, or by drawing a volume of suspension greater than the desired volume into the syringe, and removing to waste any excess suspension volume and any air bubbles present in the syringe. In another example, a 250 mg crystalline iloperidone dose is desired, contained in a suspension volume between about 1.25 mL and about 1.5 mL. This may be achieved by drawing 1.25 to 1.5 mL of suspension into the syringe, or by drawing a volume of suspension greater than the desired volume into the syringe, and removing to waste any excess suspension volume and any air bubbles present in the syringe. Other examples, which are prepared analogously to the foregoing, are provided below in Table 1. In any event, a final visual check is then performed prior to administration to ensure complete suspension of the crystalline iloperidone. If necessary, the syringe may be gently inverted, e.g., twice, to re-suspend any possible sedimentation.

TABLE 1
Exemplary formulations

| Amount of | | Concentration | | | Injection | |
|---|---|---|---|---|---|---|
| crystalline | Vehicle | of iloperidone | | Target | volume | |
| iloperidone | volume | in vehicle | | Dose | (suspension) | |
| 600 mg | 3.0 mL | 200 | mg/mL | 500 mg | 2.5 | mL |
| 600 mg | 3.3 mL | 181.82 | mg/mL | 500 mg | 2.75 | mL |
| 600 mg | 3.4 mL | 176.47 | mg/mL | 500 mg | 2.83 | mL |
| 600 mg | 3.6 mL | 166.67 | mg/mL | 500 mg | 3.0 | mL |
| 600 mg | 3.0 mL | 200 | mg/mL | 250 mg | 1.25 | mL |
| 600 mg | 3.3 mL | 181.82 | mg/mL | 250 mg | 1.37 | mL |
| 600 mg | 3.4 mL | 176.47 | mg/mL | 250 mg | 1.42 | mL |
| 600 mg | 3.6 mL | 166.67 | mg/mL | 250 mg | 1.5 | mL |

In a further embodiment, methods are provided for administering an injectable depot formulation of crystalline iloperidone or a metabolite thereof suspended in a vehicle. The injectable depot formulation may have been prepared, for example, using methods described herein, and may particularly have been prepared less than forty eight (48) hours or less than twenty four (24) hours prior to carrying out the steps described herein for administering the injectable depot formulation. More particularly, the injectable depot formulation may have been prepared using methods described herein immediately or substantially immediately prior to carrying out the steps described herein for administering the injectable depot formulation. Substantially immediately may refer to, for example, one minute, five minutes, ten minutes, fifteen minutes, or fewer prior to administration.

Administration of the depot formulation is by deep intramuscular, e.g. intragluteal, injection, e.g., using a syringe such as an 18G×1.5 inch TW (Thin Wall) (1.2 mm×40 mm) needle. The injection is performed over a period of about five (5) seconds or less. In various embodiments, the period of about 5 seconds or less may be about 4 seconds or less, about 3 seconds or less, or about 2 seconds or less.

The intramuscular injection is delivered using a single push motion, in which the syringe plunger rod is depressed in one continuous motion, and the entire dose is delivered in the period of 5 seconds or less. The single push motion is performed at a substantially steady rate of speed.

The volume of suspension to be injected over the period of less than 5 seconds contains one dose, and may be, e.g., about 2.5 mL to about 3.0 mL, or about 1.25 to about 1.5 mL. The volume administered may contain a dose of, e.g., 125-500, 250-500, about 500, or about 250 mg of crystalline iloperidone suspended in a vehicle, in a concentration of about 166.67 mg to about 200 mg crystalline iloperidone per mL of vehicle.

In a further embodiment, the foregoing methods of preparing and administering an injectable depot formulation of crystalline iloperidone may be combined, such that the methods of administration are carried out immediately after performing the methods of preparation described herein.

In a still further embodiment, an injectable depot formulation of crystalline iloperidone is provided, in which the injectable depot formulation is prepared according to the processes described above.

The skilled artisan will appreciate that additional preferred embodiments may be selected by combining the preferred embodiments above, or by reference to the examples given herein.

### EXAMPLES

### Example 1: Crystalline Iloperidone Particle Size Determination

Crystalline iloperidone particle sizes are determined in accordance with the following example. The Malvern Mastersizer 2000 (Malvern Instruments, Ltd., Malvern, UK) laser light scattering particle size analyzer and Hydro 2000 (Malvern Instruments, Ltd., Malvern, UK) wet sample dispersion unit are turned on, allowing time for the laser to warm up, e.g., for about 30 minutes. The PC is turned on, Mastersizer 2000 software is opened, and a file is created for storing results. An instrument verification check is performed according to SGS SOP EQP-525-10 Operation Preventative Maintenance and Performance Verification of the Mastersizer 2000. A new file for results storage is created, and the device is configured for Existing SOP for iloperidone. The parameters listed under the SOP are verified to confirm accuracy, namely: sample material name, iloperidone depot for injection; refractive index, 1.53, absorption, 0.1; dispersant medium, saturate 0.1% Tween 80 (Sigma Aldrich Co., St. Louis, Mo.) with 0.1% w/v sample; refractive index, 1.33; result model, general purpose; particle shape, irregular; sensitivity, normal; pump speed, 2000 RPM; sample measurement time, 12 seconds (12,000 snaps); background measurement time, 12 seconds (12,000 snaps); size range, 0.020 to 2000.000 μm; aliquot per SOP, 1; number of measurement cycles, 5 (report average); obscuration limits, 10-20%. Verification is made that the outlet tube of the dispersion unit is into the waste tank, and the tank is not full; the waste container is emptied on an as needed basis. The Quality Audit Standard is tested using the parameters in SOP EQP-525D-10, “Operation, Preventative Maintenance and Performance Verification of the Mastersizer 2000,” and the instrument verification check criterion passes per SOP EQP-525D-10.

Samples are prepared by saturating 0.1% Tween 80 (Sigma-Aldrich Co., St. Louis, Mo.) polysorbate 80 with 0.1% w/v crystalline iloperidone. 1.0 g of Tween 80 is weighed into a 1000 mL volumetric flask containing about 950 mL of E-pure water, and mixed until the Tween 80 is fully dissolved. 1.0 g of sample is then weighed and added to the same volumetric flask. The volumetric flask is stirred for 30 minutes, and sonicated for 30 minutes. The mixture is diluted to volume with E-pure water, mixed well, and filtered through 0.2 μm filter using vacuum. 200 mg of sample is transferred to a plastic 20 mL container. A few drops of Dispersant Media is added, and mixed by vortexing for 30 seconds. About 5.0 mL of Dispersant Media is added, and the sample slurry is vortexed for 30 seconds and sonicated for 15 seconds to fully disperse contents. The cell is rinsed three times with E-pure water after QAS3001B standard measurement. The cell is drained and manually filled with Dispersant Media. Stirring speed is increased to 2500 RPM, and media is allowed to circulate through the cell for at least 30 seconds. Stirring is turned off, and Dispersant Media is drained. The cell is then filled with Dispersant Media and equilibrates for at least 30 seconds. The SOP is started with the parameters for the sample so that the instrument is ready to perform measurements as soon as the sample is introduced to the Hydro 2000 unit. Background measurement is performed in automatic mode Immediately after external sonication, the sample slurry is added to the Hydro unit to achieve obscuration between the obscuration range and start measuring. Each sample is measured twice (out of one preparation).

The resulting histogram is then evaluated. If uniformity is greater than or equal to 0.9, the measurement is discarded and repeated measurements are taken, starting with the previously described step of rinsing the cell three times with E-pure water. If necessary, a new sample may be prepared. Results for the Dv50 for two sample measurements should not differ from one another by more than 25% relative difference (% RD):

$$\% RD = \frac{\vert ( A 1 - A 2 ) \vert}{( A 1 + A 2 ) / 2} \times 100 \%$$

where:

A1=result for the first replicate, and

A2=result for the second replicate.

If results for Dv50 differ more than for 25%, a third replicate is measured of the same sample. The third replicate is compared with the first two (in pairs) to confirm which replicate is out of the trend. Similar trials are used to calculate average Dv10, Dv50, and Dv90 values. According to the foregoing methods, average Dv10 values are found to be 14-50 μm, average Dv50 values are found to be 91-118 μm, and average Dv90 values are found to be 188-241 μm. The averages are calculated based on n=2 trials, where each trial is the average of n=5 acquisitions from the same measurement (average reported by software).

### Example 2: Injectability Study

An injectability study is performed in which 600 mg of crystalline iloperidone is suspended in vehicle according to procedures described herein, and injected into pork meat to assess injection characteristics. Findings are presented in Table 2 below.

TABLE 2
Injectability study using 600 mg of crystalline iloperidone

| Vehicle | | Mixing | Suspension | |
|---|---|---|---|---|
| volume | | procedure | volume* | Injection observations |
| 3 | mL | Vortex | 2.8 mL | Resistance to injection into pork meat |
| | | mixed for 30 | | observed. |
| | | seconds; held | | Blockage in needle observed. |
| | | for 15 min. | | Needle was replaced with a fresh needle and |
| | | | | injecting was re-attempted: resistance to |
| | | | | injection observed again. |
| | | | | Needle was removed from pork meat and |
| | | | | attempt was made to clear the needle block |
| | | | | with force push. Some quantity of |
| | | | | suspension was discarded when removing the |
| | | | | needle block. |
| | | | | Resistance to injection with same needle |
| | | | | again observed. |
| 3 | mL | Vortex | 2.8 mL | Resistance to injection into pork meat |
| | | mixed for 30 | | observed. |
| | | seconds; held | | Blockage in needle observed. |
| | | for 15 min. | | Needle was replaced with a fresh needle and |
| | | | | injecting was re-attempted: resistance to |
| | | | | injection observed again. |
| | | | | Needle was removed from pork meat and |
| | | | | attempt was made to clear the needle block |
| | | | | with force push. Some quantity of |
| | | | | suspension was discarded when removing the |
| | | | | needle block. |
| | | | | Resistance to injection with same needle |
| | | | | again observed. |
| 3 | mL | Vortex | 2.8 mL | Resistance to injection into pork meat |
| | | mixed for 30 | | observed. |
| | | seconds; held | | Blockage in needle observed. |
| | | for 15 min. | | Needle was replaced with a fresh needle and |
| | | | | injecting was re-attempted: resistance to |
| | | | | injection observed again. |
| | | | | Needle was removed from pork meat and |
| | | | | attempt was made to clear the needle block |
| | | | | with force push. Some quantity of |
| | | | | suspension was discarded when removing the |
| | | | | needle block. |
| | | | | Resistance to injection with same needle |
| | | | | again observed. |
| 3.3 | mL | Vortex | 3.0 mL | Resistance to injection into pork meat |
| | | mixed for 30 | | observed. |
| | | sec and held | | Blockage in needle observed. |
| | | for 15 min | | Needle was replaced with a fresh needle and |
| | | | | injecting was re-attempted: resistance to |
| | | | | injection observed again. |
| | | | | Needle was removed from pork meat and |
| | | | | attempt was made to clear the needle block |
| | | | | with force push. Some quantity of |
| | | | | suspension was discarded when removing the |
| | | | | needle block. |
| | | | | Resistance to injection with same needle |
| | | | | again observed. |
| 3.3 | mL | Vortex | 3.0 mL | Resistance to injection into pork meat |
| | | mixed for 30 | | observed. |
| | | sec and held | | Blockage in needle observed. |
| | | for 15 min | | Needle was replaced with a fresh needle and |
| | | | | injecting was re-attempted by fast push. |
| | | | | Entire suspension volume injected into |
| | | | | pork meat in less than 5 seconds. |
| 3.3 | mL | Vortex | 3.1 mL | Resistance to injection into pork meat |
| | | mixed for 30 | | observed. |
| | | sec and held | | Blockage in needle observed. |
| | | for 15 min | | Needle was replaced with a fresh needle and |
| | | | | injecting was re-attempted by fast push. |
| | | | | Entire suspension volume injected into |
| | | | | pork meat in less than 5 seconds. |
| 3.6 | mL | Vortex | 3.3 mL | Resistance to injection into pork meat |
| | | mixed for 30 | | observed. |
| | | sec and held | | Blockage in needle observed. |
| | | for 15 min | | Needle was replaced with a fresh needle and |
| | | | | injecting was re-attempted: resistance to |
| | | | | injection observed again. |
| | | | | Needle was removed from pork meat and |
| | | | | attempt was made to clear the needle block |
| | | | | with force push. Some quantity of |
| | | | | suspension was discarded when removing the |
| | | | | needle block. |
| | | | | Resistance to injection with same needle |
| | | | | again observed. |
| 3.6 | mL | Vortex | 3.3 mL | Resistance to injection into pork meat |
| | | mixed for 30 | | observed. |
| | | sec and held | | Blockage in needle observed. |
| | | for 15 min | | Needle was replaced with a fresh needle and |
| | | | | injecting was re-attempted by fast push. |
| | | | | Entire suspension volume injected into |
| | | | | pork meat in less than 5 seconds. |
| 3.6 | mL | Vortex | 3.3 mL | Resistance to injection into pork meat |
| | | mixed for 30 | | observed. |
| | | sec and held | | Blockage in needle observed. |
| | | for 15 min | | Needle was replaced with a fresh needle and |
| | | | | injecting was re-attempted by fast push. |
| | | | | Entire suspension volume injected into |
| | | | | pork meat in less than 5 seconds. |
| 3.3 | mL | Manually | 3.1 mL | Entire suspension volume was injected into |
| | | shake for 30 | | pork meat by fast push in 3 secs. |
| | | sec, no hold | | |
| | | after | | |
| | | reconstitution | | |
| 3.3 | mL | Manually | 3.1 mL | Start injecting by fast push; after injecting 1.2 |
| | | shake for 30 | | mL, rate of injection was slowed. Resistance to |
| | | sec, no hold | | injection and needle blockage were observed |
| | | after | | upon slowing. |
| | | reconstitution | | Needle was replaced with a fresh needle, |
| | | | | remaining suspension was injected by fast |
| | | | | push. |
| 3.3 | mL | Manually | 3.0 mL | Start injecting by fast push; after injecting 1.6 |
| | | shake for 30 | | mL, rate of injection was slowed. Resistance to |
| | | sec, no hold | | injection and needle blockage were observed |
| | | after | | upon slowing. |
| | | reconstitution | | Needle was replaced with a fresh needle, |
| | | | | remaining suspension was injected by fast |
| | | | | push. |
| 3.3 | mL | Manually | 3.1 mL | Entire suspension volume was injected into |
| | | shake for 30 | | pork meat by fast push in 2 secs. |
| | | sec, no hold | | |
| | | after | | |
| | | reconstitution | | |
| 3.3 | mL | Manually | 3.1 mL | Entire suspension volume was injected into |
| | | shake for 30 | | pork meat by fast push in 2 secs. |
| | | sec and held | | |
| | | for 15 min | | |
| 3.3 | mL | Manually | 3.0 mL | Start injecting by fast push; after injecting 1.0 |
| | | shake for 30 | | mL, rate of injection was slowed. Lag of |
| | | sec and hold | | approx. 1 sec. was observed. Resistance to |
| | | for 15 min | | injection and needle blockage were observed |
| | | | | upon slowing. |
| | | | | Needle was replaced with a fresh needle, |
| | | | | remaining suspension was injected by fast |
| | | | | push. |
| 3.3 | mL | Manually | 3.0 mL | Start injecting by fast push; after injecting 0.8 |
| | | shake for 30 | | mL, rate of injection was slowed. Resistance to |
| | | sec and hold | | injection and needle blockage were observed |
| | | for 15 min | | upon slowing. |
| | | | | Needle was replaced with a fresh needle, |
| | | | | remaining suspension was injected by fast |
| | | | | push. |
| 3.3 | mL | Manually | 3.1 mL | Entire suspension volume was injected into |
| | | shake for 30 | | pork meat by fast push in 4 secs. |
| | | sec and hold | | |
| | | for 15 min | | |

*Observed suspension volume in syringe after removing foam/air bubbles and priming the needle.

The observations described in Table 2 demonstrate that the preparation methods described herein, in which the concentration of crystalline iloperidone in the vehicle is 166.67 to 200 mg of crystalline iloperidone per mL of vehicle, result in a depot formulation of, e.g., a 250 mg or 500 mg dose of crystalline iloperidone that can be administered without undue resistance or clogging of the needle. The data in Table 2 further demonstrate that administration methods in which the volume of suspension is injected over a period of less than five seconds results in the successful administration of the injection volume. Still further, the observations support the use of manual shaking and vortexing to suspend crystalline iloperidone in vehicle, either in the presence or absence of a hold period following suspension.

While various embodiments are described herein, it will be appreciated from the specification that various combinations of elements, variations or improvements therein may be made by those skilled in the art, and are within the scope of the invention. In addition, many modifications may be made to adapt a particular situation or material to the teachings of the invention without departing from essential scope thereof. Therefore, it is intended that the invention not be limited to the particular embodiment disclosed, but that the invention will include all embodiments falling within the scope of the appended claims.

1. A method of preparing an injectable depot formulation of crystalline iloperidone, comprising:
combining the crystalline iloperidone with a suspension vehicle, wherein the crystalline iloperidone is present in the suspension vehicle at a concentration of 166.67 mg to 200 mg of crystalline iloperidone per mL of vehicle solution.

2. The method of claim 1, wherein the amount of crystalline iloperidone combined with the suspension vehicle is about 600 mg, and the amount of the suspension vehicle combined with the crystalline iloperidone is about 3.0 mL to about 3.6 mL.

3-7. (canceled)

8. The method of claim 1, wherein the suspension vehicle comprises, per 2 mL of suspension vehicle: poloxamer 188 in an amount of 4.00 mg, carboxymethyl cellulose (CMC) sodium in an amount of 14.00 mg, mannitol in an amount of 90.00 mg, and water in an amount of q.s. to 2 mL; and
wherein the particle size of the crystalline iloperidone is characterized by a Dv50 of up to about 120 μm.

9. The method of claim 1, further comprising:
after combining the crystalline iloperidone with the suspension vehicle, suspending the crystalline iloperidone in the suspension vehicle by agitating, vortexing, or shaking;
allowing the suspension to sit undisturbed for a fifteen (15) minute period following the suspending;
after the expiration of the 15 minute period, gently re-dispersing the suspension; and
drawing a dosage of the suspension into a syringe within twenty (20) seconds of the re-dispersing.

10-13. (canceled)

14. The method of claim 9, further comprising:
removing any excess suspension volume and any air bubbles from the syringe, wherein after the removing step, an injection volume of about 2.5 to 3.0 mL is to be administered, and a dose of crystalline iloperidone contained in the injection volume is about 500 mg.

15. (canceled)

16. The method of claim 9, further comprising:
removing any excess suspension volume and any air bubbles from the syringe, wherein after the removing step, an injection volume of about 1.25 to 1.5 mL is to be administered, and a dose of crystalline iloperidone contained in the injection volume is about 250 mg.

17. (canceled)

18. A method of administering an injectable depot formulation of crystalline iloperidone suspended in a vehicle, comprising:
using a syringe, intramuscularly injecting the depot formulation over a period of about five (5) seconds or less.

19-21. (canceled)

22. The method of claim 18, wherein the depot formulation contains crystalline iloperidone and the vehicle in a concentration of about 166.67 mg to about 200 mg crystalline iloperidone per mL of vehicle;
wherein the particle size of the crystalline iloperidone is characterized by a Dv50 of up to about 120 μm; and
wherein the suspension vehicle comprises, per 2 mL of suspension vehicle: poloxamer 188 in an amount of 4.00 mg, carboxymethyl cellulose (CMC) sodium in an amount of 14.00 mg, mannitol in an amount of 90.00 mg, and water in an amount of q.s. to 2 mL.

23. The method of claim 22, wherein a volume of the depot formulation injected over the period of less than 5 seconds is about 2.5 to about 3.0 ml, and wherein the injectable depot formulation of crystalline iloperidone contains a dose of about 500 mg of crystalline iloperidone.

24. (canceled)

25. The method of claim 22, wherein a volume of the depot formulation injected over the period of less than 5 seconds is about 1.25 to about 1.5 ml, and wherein the injectable depot formulation of crystalline iloperidone contains a dose of about 250 mg of crystalline iloperidone.

26-31. (canceled)

32. The method of claim 18, further comprising:
using a syringe, intramuscularly injecting the depot formulation over a period of about five (5) seconds or less using a single push motion performed at a steady rate of speed; and
wherein the injecting is performed less than forty-eight (48) hours after the crystalline iloperidone is suspended in the vehicle.

33-35. (canceled)

36. A method of preparing and administering an injectable depot formulation of crystalline iloperidone suspended in an aqueous vehicle, comprising:
combining the crystalline iloperidone with the aqueous vehicle to form a suspension, wherein the crystalline iloperidone and the aqueous vehicle are present in the suspension at a concentration of 166.67 mg to 200 mg of crystalline iloperidone per mL of aqueous vehicle; and
using a syringe, intramuscularly injecting the suspension over a period of about five (5) seconds or less.

37-40. (canceled)

41. The method of claim 36, wherein the suspension vehicle comprises, per 2 mL of suspension vehicle: poloxamer 188 in an amount of 4.00 mg, carboxymethyl cellulose (CMC) sodium in an amount of 14.00 mg, mannitol in an amount of 90.00 mg, and water in an amount of q.s. to 2 mL; and
wherein the particle size of the crystalline iloperidone is characterized by a Dv50 of up to about 120 μm.

42. The method of claim 36, wherein the amount of crystalline iloperidone is about 600 mg, and the amount of the aqueous vehicle is about 3.0 mL to about 3.6 mL.

43. The method of claim 36, further comprising:
after forming the suspension and prior to intramuscularly injecting the suspension, allowing the suspension to sit undisturbed for a fifteen (15) minute period; and
after expiration of the 15 minute period, gently re-dispersing the suspension.

44. (canceled)

45. The method of claim 36, further comprising drawing a dosage of the suspension into a syringe for administration; and
removing any excess suspension volume and any air bubbles from the syringe.

46. (canceled)

47. The method claim 45, wherein a volume of the depot formulation injected over the period of less than 5 seconds is about 2.5 to about 3.0 mL; and a dose of crystalline iloperidone contained in the injection volume is about 500 mg.

48. (canceled)

49. The method claim 45, wherein a volume of the depot formulation injected over the period of less than 5 seconds is about 1.25 to about 1.5 mL; and a dose of crystalline iloperidone contained in the injection volume is about 250 mg.

50-53. (canceled)

54. The method of claim 36, further comprising:
intramuscularly injecting the depot formulation over a period of about five (5) seconds or less using a single push motion performed at a steady rate of speed;
wherein the step of intramuscularly injecting is performed less than forty-eight (48) hours after the step of combining the crystalline iloperidone with the aqueous vehicle.

55-57. (canceled)

58. An injectable depot formulation of crystalline iloperidone prepared by the process of claim 1.

59-60. (canceled)
